(19) United States
(12) Patent Application Publication
(10) Pub. No.: US 2021/0233646 A1
(43) Pub. Date: Jul. 29, 2021

(54) SYSTEM AND METHOD FOR COLLOBORATIVE AND INTERACTIVE IMAGE PROCESSING

(71) Applicant: Hewel, Rouen (FR)

(72) Inventors: Gilles Le Naour, Bry-Sur-Marne (FR); Fabien Lieval, Bussy-Saint-Georges (FR)

(21) Appl. No.: 16/769,064

(22) PCT Filed: Dec. 7, 2018
(86) PCT No.: PCT/EP2018/084060

§ 371 Date: Jun. 2, 2020

(30) Foreign Application Priority Data
Dec. 8, 2017 (FR) 1771328

(51) Int. Cl. G16H 30/40; G16H 80/00; G06T 7/00; G16H 30/20

(52) U.S. Cl. CPC G16H 30/40; G16H 30/20; G06T 7/0012; G16H 80/00

(57) ABSTRACT

The invention proposes a system (100) and a method for collaborative and interactive image processing aiming to limit errors in the examination of an image. The system according to the invention comprises a distribution console (110) for distributing images to be processed (200) and for collecting annotated images capable of communicating locally with at least two processing interfaces (120) capable of displaying an image to be processed. The console and the interfaces are programmed to transmit a copy of the image to be processed to each processing interface from the distribution console, to provide tools for modifying the copy of the image on each interface so as to obtain an annotated image, to transmit each annotated image to the distribution console from each processing interface, and to generate a resulting image from the annotated images using a determined combinatorial algorithm representing the common points and the differences between the annotated images.

The present invention relates to a system and to a method for collaborative and interactive image processing, for example in the context of an industrial quality inspection or in the context of assisting with medical diagnostics or teaching.

The invention applies in particular to the industrial field (for example aeronautics or microelectronics), to the medical field and to any other field requiring the sharing of opinions and the graphical analysis of images in order to limit the risks of individual errors of judgement.

In many technical fields, analyzing graphical data in the form of images requires having several opinions and/or contributions from several experts in order to ensure good-quality analysis of the data and to then be able to make a relevant diagnosis. Consideration is of course given to the medical field, and particularly to radiological medical imaging, and to digital slides of histological examinations, in which accurate analysis with good observation quality is essential in order then to be able to make the correct diagnosis and suggest the correct treatment.

Consideration is also given to the aeronautical field, in which structural analyses have to be performed regularly in order to ensure that a particular component or assembly has not undergone a deformation or a treatment incompatible with its role in the aircraft.

It is then essential to be able to ensure that the specialist or specialists in charge of these analyses have correctly identified the areas of interest on the images of the structures, such as cancerous cells in the medical field or microcracks in the aeronautical field, and the extent thereof.

The invention also applies to the teaching field in order to allow a teacher to teach students how to analyze certain images (medical or industrial images) and to evaluate these same students with respect to objective technical criteria. The invention also aims to allow face-to-face students (that is to say those who are present in class as opposed to remote students) to easily question their teacher about particular areas of the image, in synchronous mode (that is to say at the same time) or asynchronous mode (that is to say at different times during the working session).

One example of a more specific field of application of the invention is that of pathological anatomy and cytology (“PAC” hereinafter).

This is a medical specialism that consists in looking for tissue and cell anomalies in a sample taken from an organ. The examination is based on the macroscopic and/or microscopic study of tissue or its derivatives taken from patients. Tissue or cell histology slides (hereinafter “slides”) are observed by a pathological anatomy and cytology doctor (or “PAC doctor”) who lists, quantifies and qualifies his observations in a report. Said report is transmitted to the treating doctor or clinician, who will interpret it by taking into account other medical data from other examinations, either for example in order to request a surgical inspection, or to directly suggest surgery on the basis of the first biopsy examination, or to directly make a diagnosis and suggest a treatment.

PAC is used in particular in the context of diagnosing tumors and in the customized therapeutic care and targeted treatment of cancers or certain non-cancerous diseases, but also for diagnosing inflammatory, degenerative, metabolic or infectious diseases.

In the light of the increasing number of PAC files, the fall in the demographics of PAC doctors and the emergence of a need for PAC files to be traceable, the concept of “digital pathology” emerged in the 90s, combining PAC with high-speed digitization technologies.

The development of slide scanners thus allowed this new discipline to emerge, which has become a medical examination and research tool as well as a powerful pedagogical tool.

As is known, slide scanners produce virtual slides (“virtual slides” hereinafter) that are what are termed as “high-definition” images, since they have a large number of pixels (for example from 10000×10000 pixels to 200000×200000 pixels), this being manifested in a very high number of bytes, typically of the order of several tens of megabytes to several gigabytes (for example from 10 MB to 10 GB).

Using virtual slides in PAC may have several additional uses for practitioners (PAC doctors, researchers, medical experts):
a) Individual use: a practitioner makes a medical diagnosis on one or more cases, or consults one or more type cases from case libraries in order to develop an opinion;
b) Collective use: a practitioner submits one or more cases to his peers for opinions (for example medical teams or PAC review committees) , or forms a group of other practitioners around learning cases (for example university or post-university seminars), or conducts research projects in association with his peers in relation to specific slides that may lead to scientific publications.

For all collective uses, the need very often arises to share or to compile or to be able to trace information or to organize work collaboratively. This need is not however met by modern software and/or hardware.

Specifically, several companies have developed specialist items of software for reading and analyzing virtual slides, and sometimes for assisting with diagnostics.

These items of software all have the same functionalities: creating indexes of images of virtual slides, viewing and manipulating individual images, defining regions of interest (hereinafter “ROIs”) or objects of interest (hereinafter “OOIs”), counting and annotating (that is to say commenting on the image, the OOIs or the ROIs). Some of them also offer automatic shape recognition and counting solutions based on mathematical algorithms.

Their main benefit is that of making it easier to store and broadcast the virtual slides, avoiding them being lost or damaged and saving time when annotating the images and counting the ROIs/OOIs.

However, these items of software are nowadays intended for individual use for individual uses, from practitioners' computers. They are also generally perceived to be difficult to use and not very ergonomic.

Virtual slides, and images in general, are for their part stored either on computers or on servers which can be accessed remotely via telecommunication means (for example cloud hosting). Given the size in bytes of the virtual slides, the computing devices that are used (computer and server) have to be endowed with significant hardware resources, both in terms of computational power and in terms of transmission speed. These items of software therefore depend on highly heterogeneous computing architectures of the practitioners and the available telecommunication networks, which are themselves highly variable and inconsistent. In practice, a lack of fluidity is often experienced when displaying and manipulating the virtual slides (for example latency time), this having a detrimental effect on the observation quality of the virtual slides and therefore constituting a first major challenge of the invention.

These items of software are furthermore not collaborative or interactive. They do not offer any solution for ensuring both traceability of discrepancies and the graphical analysis thereof between examinations by several PAC doctors, or solving the problem of data anonymization.

Now, at present, in the medical field and in the aeronautical field, it is common to call on networks of experts regarding the interpretation of medical images or assemblies of mechanical components in the most complex cases. The initial individual diagnosis is then enriched through the exchange with peers, until arriving at a contradictory diagnosis based on several expert opinions. However, these networks, which are often national or international, are interrogated remotely, and each expert works on his own and when he is available.

In addition, in the medical field, the requirements in terms of data anonymization make it difficult to manage the sharing of information. It is thus necessary to transmit, to each expert, data (sample, image of the sample, age, gender of the patient, etc.) in anonymized form so that he is not able to identify the patient, by providing him with an identification code accompanying the data. He then sends a report with this identification code, which report then has to be returned to the treating doctor with the correct identity of the patient. This anonymization/reidentification chain is a source of human error and should comprise numerous checkpoints. Furthermore, this chain is more complex the greater the number of experts who are consulted. In order to limit the risks, this leads to only a small number of experts, that is to say two or three, being consulted.

In spite of all this, it is not unusual for the results of a patient to be given to another patient, with all of the negative consequences that this involves.

The lack of interaction between the members of the network and the data anonymization problems constitute a second challenge of the invention.

Moreover, these items of software offer tools for annotating images with text, the definition of the terms and the implementation of which are left to the discretion of practitioners. Several practitioners consulting the same images may thus define heterogeneous OOI or ROI categories and annotation modules, which may ultimately lead to different diagnoses.

Mention may be made for example of document US20140292814, which describes a diagnosis assistance system using virtual slides, in which several practitioners are able to add text comments derived from their cognitive analysis of the image to a virtual slide image. It is thus possible to add a comment to an area of text that points to a given object in the image by virtue of a line that links the text area to the object in the image, in exactly the same way as word processing in which several authors are able to add comments in the margin, these comments being linked to a word in the text.

The claimed benefit of this system is that all of the comments can be displayed simultaneously above the image, that is to say juxtaposed and in a manner grouped together depending on the location of the object that is commented upon. The reader is therefore simply able to identity the author (or his function) depending on the display format of the text area, but he is incapable of directly deducing the meaning of the comments from the displayed image.

Specifically, on the one hand, these comments are simply superimposed on the image, which is not modified as such.

The pixels of the image that are displayed on the screen are indeed modified so as to be able to display the comments, but the pixels of the image file as such are not modified. The comments are thus displayed in superimposed form, that is to say juxtaposed with the image on the screen, and not inserted into the file of the image itself. This is also demonstrated by the embodiment in which the comments appear only when the cursor is placed over an area that is commented upon.

In other words, the annotations in that document consist only of comments superimposed on text areas and in the digital text format, whereas the image is in the digital image format. These annotations are not graphical annotations on the image, that is to say modifications to the digital image format of the pixels of the image.

Thus, in that document, the comments and the image are not in the same digital format.

On the other hand, the system in that document is not able to analyze the comments, since they are text that needs to be interpreted semantically by the reader, and this function is not able to be performed automatically by an algorithm.

Therefore, since this system is not able to semantically analyze the comments and thereby deduce their meaning, it is not able to generate a summary of these comments, for example in the form of a single comment that groups together similar opinions and various comments with different opinions. The end user is therefore obliged to read all of the comments that are juxtaposed and superimposed on the image when it is displayed (the image as such not being modified per se), interpret them semantically and summarize them intellectually.

Such a system is thus less usable the higher the number of authors and/or objects to be commented upon, because the display becomes illegible and interpretation becomes lengthy and tedious.

Furthermore, since the system in document US20140292814 leaves each author free to edit his comments, in particular in terms of the vocabulary that is used, it is absolutely impossible to interpret these comments automatically.

Allowing automatic analysis and automatic summary of this analysis is therefore one challenge of the invention.

Guiding users in graphically annotating the images is another challenge of the invention.

The present invention therefore aims to propose a collaborative and interactive tool designed for several people to work face-to-face, synchronously or asynchronously, and allowing the participants to jointly examine identical images and to share points of view and individual examinations of the images, using terminology databases and graphical annotation tools associated with identical and pre-recorded decision sequences that are specific to the context (for example pathology).

Collaborative is understood to mean that several people may study the same image at the same time, and participate in analyzing it and commenting on it.

Interactive is understood to mean that several people may participate in the processing (analysis and marking) of the same image, that is to say that several people may or may not act at the same time, independently or interactively, on the image in order to modify it or annotate it.

Graphical annotation is understood to mean modifying the pixels of the image, and therefore the data of the image, this annotation not having any semantic meaning.

The present invention also aims to make it possible to automatically compare the results of a graphical examination by experts, to analyze common points and graphical differences in order to be able to automatically establish a standardized preliminary report, thus ensuring that all of these data are traceable, and comprising statistically analyzing these graphical data.

The present invention also aims to allow one of the experts, designated as author, to supplement this preliminary report with his interpretation of the image, enlightened by the exchanges with the other participants.

By virtue of this report, the author is thus able to transmit an enlightened opinion on the case, that is to say with full knowledge of the common points and the discrepancies in the experts' analysis, and their representativeness in the panel of experts that analyzed the image or images, to the responsible parties (referring doctors, team leader, etc.), who for their part will subsequently make a diagnosis.

The present invention therefore aims to propose a collaborative and interactive diagnosis assistance tool, but the system or the method according to the invention is at no point able to establish a diagnosis itself.

To this end, the present invention proposes a collaborative and interactive image processing system comprising a console for distributing images to be processed and for collecting annotated images, able to communicate locally with at least two processing interfaces that are able to display an image to be processed, and wherein the console and the interfaces are programmed to transmit a copy of the image to be processed to each processing interface from the distribution console, so as to provide tools for graphically modifying the copy of the image on each interface in order to obtain an image annotated with graphical modifications, to transmit each annotated image to the distribution console from each processing interface and to generate a result image from the annotated images using a combinatorial algorithm analyzing the graphical modifications of each annotated image in order to determine similar graphical modifications and different graphical modifications between the annotated images, and synthesizing this analysis by generating a result image consisting of pixels of graphical formats that differ between the similar graphical modifications and the different graphical modifications.

Combinatorial algorithm is understood to mean that the algorithm evaluates the similarity between the graphical annotations of the annotated images, fuses the graphical annotations by similarity group before inserting them in the result image in a single format per group, able to be identified directly on the result image. In other words, a combinatorial algorithm according to the invention analyzes the data of the image, synthesizes this analysis by producing a single image resulting from this analysis, and then displays the result of this synthesis, such that the user directly deduces the meaning therefrom.

According to other embodiments, which may be combined with one another:
the console and the interfaces may be programmed to make it possible to mark, on the copy of the image to be processed, at least one region of interest defined by a unique identifier, a representative shape and coordinates in the copy of the image to be processed;
the console and the interfaces may be programmed to allow a display in an individual mode in which the display of each interface is independent of the other interfaces, and to allow a display in a commanded mode in which the display of a first interface commands the display on the other interfaces, the change between the individual mode and the commanded mode being reversible;
the system may comprise:
   A data distribution and collection console comprising:
      a wireless transceiver for transmitting/receiving a local wireless signal;
      a memory for storing at least one image to be processed and at least one result image;
      a programmed central unit;
   at least two processing interfaces comprising:
      a wireless transceiver for transmitting/receiving a local wireless signal compatible with the transceiver of the console for allowing a local exchange of data via the wireless signal;
      a memory for storing at least one image to be processed and at least one annotated image;
      a programmed central unit;
the central unit of the console and the central unit of each interface may be programmed to:
   transmit a copy of at least one image to be processed to all of the interfaces;
   transmit a copy of at least one image to be processed to all of the interfaces;
   display a whole image to be processed on each interface,
   zoom in and/or zoom out on the displayed image on command and store a zoom value representative of the zoom level of the displayed image with respect to the whole image and a position of the displayed image with respect to the whole image;
   execute, from a first interface, a display in a reversible commanded mode of displaying the other interfaces, by commanding a display identical to the display of the first interface on all of the other interfaces;
   affix at least one graphical annotation mark on the image to be processed in order to generate an annotated image;
   record and transmit each annotated image to the console with an identifier representative of the interface that transmitted the annotated image;
if the display in commanded mode is activated by a user, the central unit of the console and the central unit of each interface may be programmed to store an initial display of each interface beforehand, to command a display identical to the display of the first interface on all of the other interfaces, and then to redisplay the initial display of each interface when the commanded mode is deactivated;
the central unit of the console may be programmed to combine the annotated images sent by each interface and generate a result image with a combinatorial algorithm analyzing the graphical modifications to each annotated image in order to determine similar graphical modifications and different graphical modifications between the annotated images, and synthesizing this analysis by generating a result image consisting of pixels of graphical formats that differ between the similar graphical modifications and the different graphical modifications;
the central unit of the console may be programmed to generate a result image in which graphical annotation marks on the annotated images having a similarity level greater than a determined similarity threshold are displayed in a first graphical format, and in which graphical annotation marks on the annotated images having a similarity level less than the determined similarity threshold are displayed in at least one second graphical format;
the central unit of the console may be programmed to execute statistical analysis of the graphical annotation marks;
the statistical analysis may comprise calculating a percentage of graphical annotation marks having a similarity level greater than a determined similarity threshold and calculating a percentage of graphical annotation marks having a similarity level less than the determined similarity threshold;
the similarity threshold may be settable;
the central unit of the console and/or of each interface may also be programmed to generate a table of correspondence between each graphical annotation mark, defined at least by position coordinates in the annotated image, and its author;
the central unit of the console may also be programmed to display an author identifier for each graphical annotation mark on the result image;
the central unit of each interface may be programmed to be able to be activated with an administrator code or with a user code, the central unit of the console being programmed to display, on an interface activated with the administrator code, administration tools comprising: a list of images to be processed that are present in the memory of the central unit, a list of identifiers of the participants, a list for descriptors of objects or of regions of interest which can be selected by users, each descriptor being associated with a different predefined graphical annotation mark;
the console may comprise a connection port for connecting to a removable memory connected to the central unit of the console;
the console may comprise a network connection port connected to the central unit of the console, said console being programmed to communicate with a remote server via a communication network in order to download at least one image to be processed and to store it in memory;
the remote server may comprise a first memory for storing original images each associated with confidential identification information, a second memory for storing corresponding images to be processed, without confidential identification information but each associated with a unique identifier, and a table of correspondence between the original images and the images to be processed;
the central unit of the console may be programmed to add, to the result image obtained after annotating an image to be processed, the unique identifier of the image to be processed, and to transmit the result image provided with the unique identifier to the remote server;
the central unit of the console and the central unit of the interfaces may be programmed to display a sequential annotation guide accompanied by a terminology database, to sequentially record the responses of each expert, and to generate a preliminary examination report comprising an examination layout for the image to be processed in which the responses of each expert are reported and combined sequentially;
the preliminary examination report may comprise a unique identifier of the image to be processed, the result image, and a standardized record of statistical analysis of the result image;
the system may comprise at least two consoles for distributing images to be processed and for collecting annotated images that are able to communicate with one another, the consoles being programmed to have a settable status allowing the consoles to be organized hierarchically; and/or
one of the consoles may be programmed to have a primary status, whereas the other consoles have a secondary status, the console having a primary status being able to command the consoles having a secondary status.

“Graphical format” is understood to mean the visual format in which an annotation is shown; this may essentially be a color, a shape (square, circle, cross, etc.), a contour line (dotted, unbroken line, line of triangles, line of squares, etc.). The graphical format should not be confused with the “digital format”, which is the format of a file which can be interpreted by a computer: for example the digital text format (txt, doc, etc.) and the digital image format (bpm, jpeg, tiff, etc.).

Another subject of the invention is a collaborative and participative image processing method comprising the following steps:
placing an image to be processed in a memory of a console able to communicate locally with at least two processing interfaces;
transmitting a copy of the image to be processed to each processing interface from the distribution console;
on each processing interface, displaying tools for graphically modifying the copy of the image to be processed in order to allow a user to graphically modify the copy of the image to be processed and thus generate an annotated image;
transmitting each annotated image to the distribution console from each processing interface, and
using the distribution console to generate a result image from the annotated images using a determined combinatorial algorithm analyzing the graphical modifications to each annotated image in order to determine similar graphical modifications and different graphical modifications between the annotated images, and synthesizing this analysis by generating a result image consisting of pixels of graphical formats that differ between the similar graphical modifications and the different graphical modifications.

According to other embodiments, which may be combined with one another:
the graphical annotations of the annotated images having a similarity level greater than a determined similarity threshold may be marked in a first graphical format in the result image, and the graphical annotations of various images having a similarity level less than the determined similarity threshold may be marked in at least one second graphical format in the result image;
the method may furthermore comprise the following steps:
   allowing a display in an individual mode in which the display of each interface is independent of the other interfaces, and
   allowing a display in a commanded mode in which the display of a first interface commands the display on the other interfaces, the change between the individual mode and the commanded mode being reversible.

Other features of the invention will be outlined in the detailed description below, given with reference to the appended drawings, in which, respectively:

FIG. 1 shows a schematic view of a first embodiment of a collaborative and interactive image processing system according to the invention in which all of the displayed images are identical;

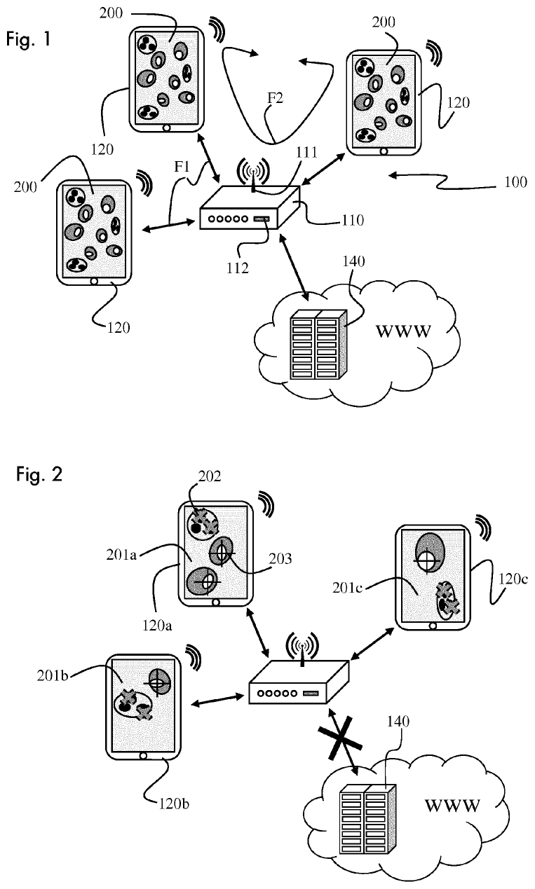

FIG. 2 shows a schematic view of a collaborative and interactive image processing system according to the invention in which the displayed images are different from one interface to another;

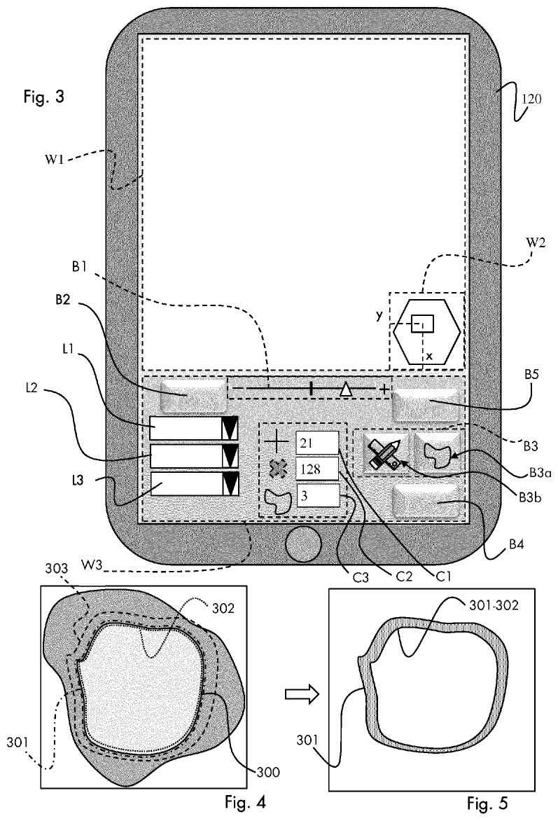

FIG. 3 shows a schematic plan view from above of an example of a display on a processing interface according to the invention;

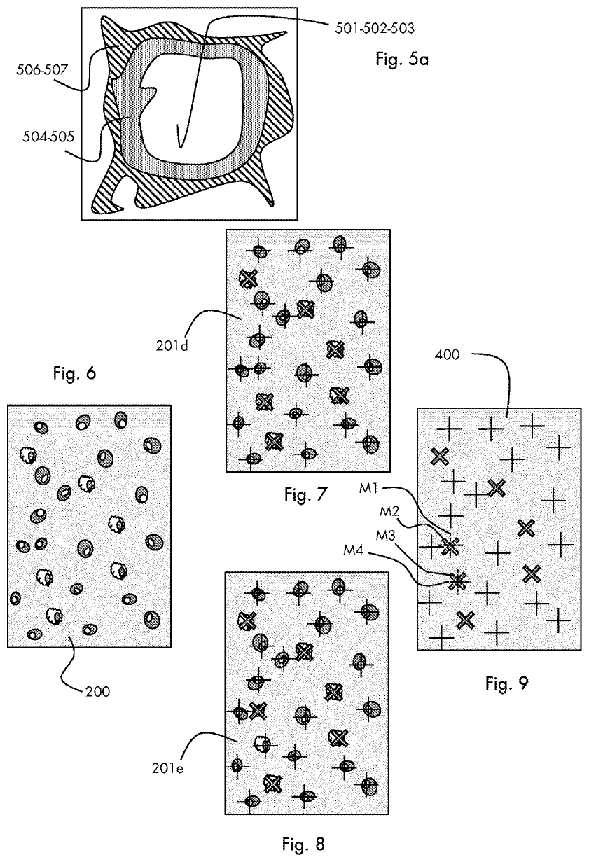

FIGS. 4 and 5 respectively show schematic plan views from above of an image on which an object of interest has been annotated by two different users and of an image displaying the difference in the annotations between the two users for the purpose of statistical analysis;

FIG. 5a shows a schematic plan view from above of an image displaying the difference in annotations between several groups of users for the purpose of statistical analysis; and

FIGS. 6 to 9 respectively show schematic plan views from above of an image to be processed, of an image annotated by a first user, of an image annotated by a second user and a result image generated by the console.

One exemplary embodiment of an image processing system according to the invention is illustrated in FIG. 1.

The system 100 comprises a console 110 for distributing images to be processed and for collecting annotated images, and at least two processing interfaces 120 able to display an image to be processed 200.

The console 110 is able to communicate locally with the interfaces 120, as illustrated by the arrows F1.

More precisely, the console 110 comprises a wireless transceiver 111 for transmitting/receiving a local wireless signal, advantageously a Wi-Fi, LoRa or SigFox signal. It also comprises a storage memory (not illustrated) for at least one image to be processed, for at least one result image and preferably for the annotated images.

It furthermore comprises a central unit (not illustrated) programmed to implement the image processing method according to the invention.

The console preferably also comprises a connection port 112 for a removable memory, the port being connected to the central unit of the console. This makes it possible to example to download images from a USB port.

The console 110 may also comprise a network connection (either wireless or wired by way of an Internet connection port connected to the central unit of the console) in order to be connected to a remote server 140 via a communication network, such as the Internet. The central unit of the console 110 is then programmed to communicate with the remote server 140 in order to download at least one image to be processed and to store it in memory.

This may preferably be an NAS (network attached storage) mini router without a fan, transmitting a local and stable Wi-Fi, LoRa or SigFox signal. This type of console is portable and therefore able to be moved easily and makes little noise, such that the working session is not disrupted.

Preferably, when the console 110 comprises an Internet connection, the central unit of the console 110 is programmed to interrupt this connection during the image processing, and to reestablish this connection only during the secure transfer of the result image and/or of an examination report to the remote server 140. By virtue of this, the connection is made in advance when preparing the working session, and is then interrupted, such that group work does not depend on the quality of the communication with the server.

The processing interfaces 120, or processing terminals, are advantageously touchscreen tablets, preferably equipped with styli for allowing the images to be annotated directly on the screen for greater accuracy.

As an alternative, they may be portable computers. In this case, the images are annotated by way of a mouse or of a touchpad. What is important is that the user is able to manipulate the images (move, zoom in/zoom out, annotate them), and communicate with the console 110.

For this purpose, the interfaces 120 comprise a wireless transceiver (not illustrated) for transmitting/receiving a local wireless signal compatible with the transceiver 111 of the console 110 in order to allow a local exchange of data via the wireless signal (arrows F1). Each interface 120 also comprises a storage memory for at least one image to be processed and for at least one annotated image, and a central unit.

The local communication is stable and of good quality. It may furthermore be encrypted and allow the work to be secured, since said work is performed “offline” without the risk of the results being hacked.

Preparation of the Working Session

A working session is prepared and led by a user called “administrator”. This may or may not be one of the experts who will participate in the working session.

In the system according to the invention, the central unit of each interface is programmed to be able to be activated with an administrator code or with a user code.

Before a working session, the person in charge of the working session enters an administrator code on one of the interfaces, this having the effect of connecting the interface to the console and making it possible to access the administration tools of the console. The central unit of the console is programmed to display, on the interface activated with the administrator code, the administration tools comprising: a list of images to be processed that are present in the memory of the central unit (or in an external memory connected to the console or in a memory of a remote server), a list for the names of the participants, a list for descriptors of objects and/or of regions of interest which can be selected by users and each associated with a different predefined annotation mark (of different shapes, of different line formats, of different colors, etc.), a list of pre-recorded types of examination or a module for recording a new type of examination prepared by the administrator.

With regard to the images, the virtual slide or slides (or other types of image) in their original proprietary format (each slide scanning company has its own image format) are routed by the administrator to the distribution console, either using a USB key or by virtue of a remote connection to the remote storage server 140.

The distribution console 110 then automatically converts the images into a standard format, advantageously the JPEG format.

It thus generates an image base, stored locally in the memory of the console 110, and a list of these images in order then to make it possible to select the images and process them on the interfaces 120.

In a first embodiment, each image remains whole and therefore comprises just a single image to be processed, a copy of which will be sent to each interface for processing. This embodiment is preferably reserved for small images in order to limit the transfer time between the console 110 and the interfaces 120.

The invention also proposes one advantageous embodiment for very large images, comprising a large number of pixels, in order to reduce the transfer time and improve fluidity.

In this embodiment, only one copy of one portion of each base image is sent to each interface.

To this end, the distribution console 110 divides each base image into a series of tiles (for example of size 256 pixels×256 pixels or 512 pixels×512 pixels). The console 110 associates a map grid with each image (and therefore with each tile) in order to generate a unique pair of (x,y) coordinates for each pixel in the image so as to ascertain the location of each tile in the initial image. The details of the process will be described below.

This makes it possible to reduce the amount of data exchanged between the console 110 and the interfaces 120 when a user zooms in on part of the image, thereby making the manipulation of the images (movements, zooming, etc.) as fluid as possible.

The administrator then also fills in the list of participants so that each image annotated during the working session is able to be associated with the expert who annotated it.

Next, the administrator prepares the list of objects of interest and/or regions of interest that will have to be identified by the participants. Either the descriptors form part of a pre-established list, or the administrator has an editing tool for creating new descriptors. He then associates an annotation mark with each object of interest and/or region of interest.

In one preferred embodiment, the previous step is performed automatically, as the administrator is able to associate a predefined type of examination with each image beforehand.

Each type of examination is associated with a list of OOIs and/or ROIs and their corresponding annotation marks.

By virtue of this, for each image associated with a type of examination, the users will have predefined graphical annotation tools. Thus, for a given examination, they will have to graphically define areas of interest and/or add a graphical mark to predefined objects of interest, such as for example the presence of an inflammatory infiltration by viewing lymphocytes or of hemorrhagic areas through the presence of hematites, etc. A predefined graphical annotation mark (for example a free surface, a cross, a square, a circle, a triangle, in unbroken or dotted lines, different colors, etc.) is associated with each descriptor.

Advantageously, each type of examination is also associated with:
a sequential annotation guide; and, preferably,
a terminology database.

A sequential annotation guide is a set of questions and/or indications and/or instructions that sequentially, that is to say step by step in a given order, guide each expert in order to correctly perform his examination. The sequence of the questions and/or indications and/or instructions may advantageously be determined by a pre-structured decision tree. In other words, a question/indication/instruction may depend on the action taken by the expert in response to a previous question/indication/instruction.

Thus, for each image associated with a type of examination, the users will have to respond to a certain number of questions and/or indications and/or instructions displayed sequentially and dynamically (that is to say depending on their response to the previous question). For example, for a given examination, the experts will have to answer a question about the regularity and/or the extent of the contours of the observed cells, about their grouping or their dispersion, etc.

The system also makes provision for the experts to be able to have a terminology database available to them (for example with reference to a specific ontology in the medical field). This is a database containing terminology entries and associated information. This is a dictionary that ensures that the experts use the same terms for the same concepts at identical zoom levels for their comments.

Thus, during the examination, the central unit of the console and the interfaces are programmed to display the sequential annotation guide accompanied by the terminology database with the copy of the image, to sequentially record the responses of each expert, and to generate a preliminary examination report comprising an examination layout for the image to be processed in which the responses of each expert are sequentially reported and combined.

In addition to the benefit of avoiding forgetting examination steps and using uniform language while guiding the experts in their examination during the working session, using a sequential annotation guide and a terminology database makes it possible to generate a complete preliminary report from all of the data generated by each expert, without setting some of these data aside because they were not foreseen. In this respect, a “general comments” section provided in the annotation guide may allow an expert to make an observation that was not foreseen in the examination sequence and that could be relevant, while at the same time being taken into account in the preliminary report.

Advantageously, the types of examination are pre-recorded and available either in the memory of the console 110 or online in a dedicated database.

The sequential annotation guides and the terminology databases may thus be prepared and designed by professional authorities and thus ensure standardization of the examinations.

As an alternative, the type of examination may be prepared by the administrator himself. In this case, he will himself define:
Optionally: a sequential annotation guide and a terminology database
The descriptors (that is to say the OOIs and/or the ROIs) that he wishes to make available to the experts, and he will associate them with a given graphical annotation mark (for example a color of a freeform area to be defined, a cross, a square, a circle, a triangle, in unbroken or dotted lines, etc.).

In both cases (pre-established examination or examination prepared by the administrator), the system is capable of counting the marks on each OOI or ROI while storing the number of annotation marks made by each expert.

The system according to the invention is furthermore also programmed to associate, with each annotation mark, at least one pair of x, y coordinates in the image and an identification of the expert who made it. The central unit of the console and/or of each interface is/are thus programmed to generate a table of correspondence between each mark, defined by its position coordinates in the annotated image, its type (cross, square, circle, triangle, unbroken or dotted lines, color of a free area to be defined, etc.), possibly its extent (in terms of number of pixels), the OOI or the ROI that it represents, and its author.

Once the session has been prepared, the administrator may end his administrator session and, if he is participating in the examination, connect with a conventional user code.

If the images have been downloaded from a remote server, the central unit of the console 110 is advantageously programmed to interrupt the network connection before the examination (as illustrated in FIG. 2), thereby making it possible to ensure firstly that there is no unauthorized connection during the examination and secondly that the work is perfectly fluid, independently of the quality of a network connection.

Sequence of the Working Session

Only users provided in the list created by the administrator are able to access the image base, using at least one identifier and a password. Other access security means may be used, such as retinal identification or fingerprints.

The communications between the console and the interfaces are furthermore preferably encrypted.

One example of a display on an interface 120 is illustrated in FIG. 3.

A first area W1 is reserved for displaying the image per se. An area W2, smaller than W1, shows the whole image of the analyzed sample (outlined by a hexagon in FIG. 3), and the portion of the image displayed in the area W1 with its location in terms of x, y data in the whole image. The user is thus able to know, at all times, where the portion that he is viewing is located with respect to the rest of the sample.

A third area W3 groups together various data and interaction buttons required for the examination.

For example, the area W3 comprises a zoom indicator B1 of the displayed image with respect to the whole image.

The area W3 furthermore comprises a connection button B2 either for ending the session or for changing to administrator mode in order to add, for example, a descriptor to be made available to the experts for the examination.

The area W3 also comprises a drop-down list L1 of the images to be displayed and a drop-down list L2 of the regions of interest to be placed on the image and to be examined. These two lists were selected by the administrator during the phase of preparing the working session.

The area W3 also comprises a graphical annotation tool area B3, comprising for example a button B3a for activating a toolbar for defining a region of interest and a button B3b for activating a toolbar for various graphical marks for annotating objects of interest with their descriptor.

The display area W3 also comprises counters C1, C2 and C3 for counting the annotations (marks and ROIs). In the illustrated example, 21 straight crosses (corresponding to a first type of OOI) have been placed, 128 oblique crosses (corresponding to a second type of OOI) have been placed, and 3 regions of interest have been defined.

Lastly, the display area W3 comprises a button B4 for saving the work that has been performed. As an alternative or in combination, the central unit of each interface 120 may be programmed to automatically save the annotated image at regular intervals. The manual or automatic saving is performed locally in a memory of the interface 120 and/or remotely, in a memory of the console 110.

Advantageously, the area W3 also comprises a scrolling menu L3 displaying specific annotation modules dedicated to particular pathologies.

Annotation

According to the invention, the console 110 and the interfaces 120 are programmed to allow a copy 200 of the image to be processed (the whole base image or a tile, that is to say a portion of the whole base image) to be transmitted to each processing interface 120 from the distribution console 110 in order to allow the copy of the image to be processed to be graphically modified on each interface 120 in order to obtain an annotated image 201a, 201b, 201c.

A low-resolution and small copy of the whole base image (a thumbnail, and therefore very small) is transmitted to each interface and displayed in the display area W2. This copy constitutes a layout of the base image to be processed.

When the base image is small, a copy of the whole base image is transmitted to each interface 120 for display in the area W1 and for annotation and examination.

The user is then able to zoom within the whole image displayed in area W1.

When the base image is large, it is divided into tiles, forming a plurality of images to be processed. In this case, a copy of a single tile to be processed, defined by default, is transmitted with the layout and displayed whole in the area W1.

Either the user annotates this tile and is able to move therein or zoom into it, or he attempts to move in the whole image. In this case, depending on the zoom level in the image and on the movement performed by the user from his interface 120, the interface produces a request that is sent to the console 110. This comprises at least: the reference (x,y) coordinates and a pair of dimensions in terms of x and in terms of y (in terms of number of pixels). The console 110, following the request, transmits the corresponding tile or tiles to the interface, which displays said tile or tiles whole in the area W1. The user is then able to zoom in on these new tiles, move between them and annotate them.

Each expert views a copy of the image to be processed (the whole image or at least one tile) on his interface 120, and he is able to manipulate it (move in the image, zoom in on it or zoom out on it) and annotate it graphically (that is to say modify the pixels forming it).

This work, although it is performed at the same time by all of the experts, is also performed independently by each expert. Thus, as shown in FIG. 2, the console and the interfaces are programmed to allow a display in an individual mode in which the display of each interface is independent of the other interfaces: the expert manipulating the interface 120a annotates a part 201a of the image, the expert manipulating the interface 120b annotates a part 201b of the image, and the expert manipulating the interface 120c annotates a part 201c of the image.

The graphical annotation marks provided in the illustrated example are oblique crosses (X) 202 and straight crosses (+) 203. These marks replace the pixels of the image at which they are positioned.

Annotation marks may be placed in two modes:
a) A simple mode in which the administrator has defined OOI categories and the users add graphical annotation marks to the elements of the image based on these categories. This is an operation of counting OOIs (and/or ROIs) and/or of locating the OOIs (and/or ROIs) and/or of evaluating the extent of the OOIs/ROIs defined by each expert.
b) A guided mode in which, in addition to the various OOI and/or ROI categories, the administrator defines or chooses a sequential annotation guide comprising sequences of annotations following the logic of a decision tree.
   The central unit of the console and the interfaces are programmed to display, with the copy of the image, a sequential annotation guide accompanied by a terminology database, in order to sequentially record the responses of each expert.
   Each user thus responds to closed questions defined by the sequential annotation guide. Depending on each response, the console or the interface (depending on the chosen implementation of the system according to the invention) determines which OOI/ROI marks to display in the toolbars B3a and B3b, depending on the corresponding sequence.
   The user then places his graphical annotation marks during each sequence, thereby modifying the pixels of the image, and continues with the following question. All of the categories are thereby not able to be accessed at the same time. The user is thus guided in his diagnosis.

By virtue of this mode of operation, the terminology and the examination sequences can be produced and confirmed by professionals (experts in the field) before any examination. This ensures that the diagnosis steps are homogenized and the differences in diagnosis modes between groups of practitioners are minimized (the final record may compare the differences in diagnoses between users who followed a homogeneous and comparable methodology on identical images).

Annotations may be produced from each interface (annotation marks for the ROIs and/or OOIs, the path followed in the decision tree, the copying of a screen and the timestamping of all of the data) freely or in an imposed manner; by imposing for example a specific area to be marked for each user. Each ROI denoted by the various experts is identified by its author, a unique number and the image in which it is located in order to ensure perfect traceability. The annotations are then sent to the console, that is to say that the (x; y) position of each pixel of the annotation is sent to the console. Annotations may be produced in imposed mode, when face-to-face, synchronously (simultaneously by all of the users) or asynchronously (by all of the users but at different times during the working session).

Timestamping makes it easier to be able to trace the actions of all of the users, with their evolution over time (potential possibility to produce annotations several times). This is useful for consolidating the results and comparing the evolutions (for example even if the image is submitted to the same experts at two different times).

The annotations of each interface are counted according to type and their location is recorded using at least one pair of x, y coordinates in the image.

The annotated image is then recorded and transmitted to the console 110.

Advantageously, the central unit of each interface is programmed to generate a table of correspondence between each annotation mark, defined at least by position coordinates in the annotated image, and its author. The type of annotation and the annotation sequence preferably also serve to define each annotation mark. Thus, when the annotated image is recorded, it is in fact only this correspondence table, representative of the annotated image, that is transmitted to the console 110. Said console may then replace the pixels of each annotation mark on the result image that will be generated, thereby limiting the volume of data transmitted and therefore contributing to fluidity.

“Annotated image” in the present invention is thus understood to mean either the annotated image as such (for example in the jpeg format) with its graphical annotation marks or a table of graphical annotation data representative of the annotated image.

Display Sharing

By virtue of the system according to the invention, it is possible to allow true cooperation between experts during the examination.

To this end, the console and the interfaces are programmed to allow a display in a commanded mode in which the display of a first interface commands the display on the other interfaces, the change between the individual mode and the commanded mode being reversible.

The commanded display according to the invention allows several users to communicate with one another via the console, as outlined by the arrow F2 in FIG. 1.

Thus, an expert wishing to attract the attention of the other experts or wishing to ask a question is able to show the other experts a specific area of the image on their own interface.

In order to activate/deactivate the commanded mode, the interface comprises a button B5 which can be selected directly by a user on the screen of the interface 120.

Only the display is commanded, the other functionalities of the system remaining available on each interface. Thus, during a display commanded by a first interface, it is possible for the users of the other interfaces to benefit from the opportunity to graphically annotate the portion of the image that is displayed.

This reversible commanded display functionality has a dual benefit:

The first one is that, during the commanded display, only the coordinates and the zoom level are sent to the other interfaces, and not the image portion as such, thereby ensuring a perfect responsiveness of the commanded display and perfect fluidity.

Each user furthermore remains in control of annotating his image. In other words, even if the same image portion is displayed on each interface, the image itself remains specific to the interface and therefore to the user and can therefore be annotated independently of the other interfaces.

The user who activated the commanded display is only able to attract the attention of the other users to a particular area of the image, without however commanding or forcing the annotation of their own image. It thus remains possible to analyze the results of each user without statistical bias, even if the commanded display function was activated during the collaborative examination of the image.

According to one advantageous embodiment, if the display in commanded mode is activated by a user, the central unit of the console and/or the central unit of each interface are programmed to store an initial display of each interface beforehand and to command a display identical to the display of the first interface on all of the interfaces.

The initial display advantageously consists of a zoom value representative of the zoom level of the displayed image with respect to the whole image (that is to say a scale in terms of real value that interacts with the zoom level) and a position of the displayed image with respect to the whole image in the form of a pair of x, y coordinates.

Thus, when a first user takes control by activating the commanded mode, the other users do not have to worry about losing the position or the zoom level of the image that they were viewing at the time of the change to commanded mode.

When the commanded mode is deactivated, the central unit of the console and/or the central unit of each interface are programmed to redisplay the initial display of each interface. Each user then returns to the display as it was before the commanded mode was activated and is able to continue his work.

This redisplaying of the initial position may be automatic or upon request from each user in order to leave them time to annotate the displayed image portion before returning to the part that they were annotating before the commanded display.

This mode of operation makes it possible to share images and points of view at the same time. The user who took control is able to directly show the other users the image area causing him a problem or to which he wishes to attract their attention.

Furthermore, the small amount of information that is transmitted (x, y coordinates of the pixels of the annotations and zoom value) allows a very fluid and fast navigation and display.

Once the work is finished and image has been fully graphically annotated and possibly commented upon in accordance with the sequential annotation guide and the terminology database, the console 110 and the interfaces 120 are also programmed to allow each annotated image to be transmitted (automatically or manually upon instruction from the users) to the distribution console 110 from each processing interface 120. As described above, this may be the annotated image per se, but it is preferably a table of annotation data representative of the annotated image, and comprising the correspondences between each annotation mark, defined by the type of annotation (according to its descriptor for example) and position coordinates in the annotated image, and its author. Such a table comprises far less data than an image, such that the transfer takes place very quickly, and several experts are able to transfer their annotations at the same time.

Generation of the Result Image

After receiving the annotation data (annotated images or tables of annotation data representative of each annotated image), the central unit of the console 110 is also programmed to generate a result image from the annotated images using a determined combinatorial algorithm for identifying (that is to say illustrating) the common points and the differences between the annotated images.

Generally speaking, the combinatorial algorithm analyzes the graphical modifications of each annotated image in order to determine similar graphical modifications and different graphical modifications between the annotated images. It then synthesizes this analysis and generates a result image consisting of pixels of graphical formats that differ between the similar graphical modifications and the different graphical modifications.

One simplified example is illustrated in FIGS. 4 and 5.

In this example, a region of interest 300 is illustrated in an unbroken line. A first user has defined this region of interest using a representative shape 301 on his interface, illustrated in a dotted line, a second user has defined this region of interest using a shape 302 on his interface, illustrated in a dotted and dashed line, and a third user has defined this region of interest using a shape 303 on his interface, illustrated in a dashed line.

The central unit of the console is programmed to generate a result image, preferably of a format identical to the image to be processed (that is to say of the same format as the annotated images), in which the annotation marks (that is to say the pixels of the annotation marks) of the annotated images having a similarity level (for example in terms of shape, in terms of position, in terms of type of mark) greater than a determined similarity threshold are fused and displayed in a first graphical format (lines, colors, line format, location), and in which the annotation marks of various images having a similarity level less than the determined similarity threshold are fused and displayed in at least one second graphical format.

In the example, illustrated in FIG. 5, the regions of interest 301 and 302 of the first and second experts have been fused and grouped together in the form of a single first line 301-302, since they have a very close similarity level: the experts have correctly defined the area of interest 300. Even if there is a slight difference between the two of them, this is not of the kind that changes the examination. The ROIs 301 and 302 have a similarity level greater than a determined similarity threshold.

The similarity threshold may be determined either by the administrator or preset in the system. As an alternative or in combination, the similarity threshold may be settable, before or after the result image is generated, for example when the latter comprises too many different marks on account of an excessively high similarity threshold that does not make it possible to adequately group together the marks of the various experts.

The ROI 303 defined by the third expert has a level of similarity with the other two ROIs 301 and 302 that is less than the determined similarity threshold. It is therefore displayed in the form of a second line at a distance from the first.

The line representing similar ROIs may be calculated and placed, for example, at the average position of the positions of the similar lines. As an alternative, it may be chosen as being one of the similar lines. The same applies for the line of the non-similar ROIs. As an alternative, each non-similar line may be displayed at the position at which the user created it.

The combinatorial algorithm within the meaning of the invention therefore consists in fusing the graphical annotations by similarity group (that is to say the graphical annotations that are identical or whose difference is less than the determined similarity threshold) and in inserting them into the result image in the form of pixels of graphical formats that differ between each group but of the same graphical format in the same group. Graphical format is understood to mean a color or a combination of shapes (for example a dashed line, dotted line, line of triangles, an unbroken line, etc.).

In other words, in order to generate the result image, the algorithm starts from the image to be processed, analyzes the similarity between the graphical annotations of the various experts based on their annotated image and forms similarity groups, and then synthesizes its analysis in the form of a result image in which the original pixels of the image are replaced automatically with pixels of specific graphical formats which can be identified directly by an observer observing the result image. For example, in order to automatically select a graphical format which can be identified directly by an observer, the algorithm may comprise a step of analyzing the spectrum of the pixels of the image or the area of the image in which the result of the combination should be displayed, and a step of selecting a color having a contrast level greater than a threshold contrast level with respect to the pixels of the image or of the analyzed image portion.

Specifically, the algorithm compares the pixels between two annotation marks and classifies them into two categories: those that are identical (that is to say that the same pixel is modified by the experts) and those that are different (that is to say that the experts have not modified it in the same way).

The algorithm then determines whether the different pixels are spaced from the identical pixels by a threshold number of pixels that determines the similarity threshold. For this purpose, it is possible to use a window of n pixels to the side that sweeps over the perimeter of the area of the identical pixels and to modify the classification of the different pixels if they are within the window (they then fall into the category of identical pixels). The pixels that remain outside of the window are still considered to be different.

Preferably, a classification step is added, consisting in calculating the percentage of different pixels remaining with respect to the number of identical pixels. If this percentage is less than a threshold percentage, the different pixels are classified as identical pixels and the marks of the two users are considered to be identical, even if they are not placed strictly at the same location on the image. If this percentage is greater than the threshold percentage, the identical pixels are displayed in a first graphical format and the different pixels are displayed in another graphical format.

This of course applies for the marks of more than two users. In this case, the similar pixels are fused by similarity groups and displayed in as many graphical formats as there are groups of pixels.

As an alternative or in combination, the analysis may be reiterated for the pixels classified as different in the first phase, in order to determine at least two groups classified by similarity within these pixels. This thus results in the difference being qualified, that is to say graphically revealing several groups of differences (see the comment on FIG. 5a).

The annotations from various experts are thus combined graphically into a single image and not simply juxtaposed, which would have a detrimental effect on the evaluation of the analysis of the image, in particular when the number of objects or regions of interest and/or experts is high.

The single result image generated by the system therefore graphically and directly gives the user, who does not need to interpret the annotated images, at least three types of information:
there are or are not analysis differences between the experts;
which experts do not agree (the user does not have to determine himself whether or not the experts agree in their graphical analysis of the image);
spatial information about the differences and the common points: their location, their shape.

In one particular embodiment, the algorithm may define several groups of differences. It is thus possible to qualify the differences, that is to say reveal several groups of graphical annotations: the common pixels, a first crown of pixels with a second similarity level, a second crown of pixels with a third similarity level, etc. This makes it possible to graphically reveal the dispersion of the experts' opinions or a hierarchy of the experts' disagreements: for example: similar, almost similar, different, very different.

One exemplary application, illustrated in FIG. 5a, relates to defining the extent of a tumor before an operation.

After analysis and synthesis by the combinatorial algorithm, the generated result image reveals that the users 501, 502 and 503 defined the tumor in a first central area with a small extent. The users 504 and 505 for their part considered that the tumor extends slightly further. Lastly, the users 506 and 507 considered that the tumor extends even further.

The system according to the invention therefore immediately attracts attention to the fact that the experts do not agree in their analysis. The user also deduces directly from the result image that some users (506-507) defined a tumor whose shape is highly complex and that may therefore radically change future treatment. The system therefore generates data that the end user would not have been able to deduce or would have been able to deduce only with great difficulty from his analysis of the modified images, especially if the number of regions of interest and/or the number of experts is high.

The system therefore makes it possible simultaneously to save precious time, to address the graphical analyses of various experts graphically and not semantically, and to improve the relevance of their future diagnosis and of the chosen treatment.

FIGS. 6 to 9 illustrate another example of obtaining a result image regarding objects of interest marked by different experts using the system according to the invention.

FIG. 6 shows the image to be processed 200 sent to all of the interfaces. FIG. 7 shows the image 201d annotated by a first expert, and FIG. 8 illustrates the image 201e annotated by a second expert. Finally, the image 9 shows the result image 400 generated by the system according to the invention.

In the result image, the annotation marks of the images annotated by the first and the second expert having a similarity level (in terms of position and in terms of type of mark) greater than a determined similarity threshold are shown in a first graphical format, here in unbroken lines. The annotation marks M1, M2, M3 and M4 having a similarity level less than the determined similarity threshold are shown in at least one second graphical format, here in dotted lines.

The display method according to the invention preferably comprises displaying the result image and dynamically displaying the name of the author of each annotation in superimposed form when a mouse cursor is moved over the image. Advantageously, the dynamic display concerns only the annotation marks M1, M2, M3 and M4 having a similarity level less than the determined similarity threshold.

The dynamic display is made possible by virtue of the correspondence table, generated by the system according to the invention (by the central unit of the console and/or of each interface), between each annotation mark, defined at least by its position coordinates in the annotated image, and its author.

The central unit of the console is also programmed to execute statistical analysis of the graphical annotation marks.

For example, the statistical analysis comprises calculating a percentage of annotation marks having a similarity level greater than a determined similarity threshold and calculating a percentage of annotation marks having a similarity level less than the determined similarity threshold.

This result makes it possible to indicate whether or not an image has a strong examination consensus between the experts, and to do so very quickly and directly even if there is a large number of marks and/or a large number of experts. This analysis may be more refined and performed only within the divergent annotation marks. It is then possible to see whether a discrepancy is a simple annotation error: for example, for a given OOI (identified by its x, y coordinates) and an expert panel of 20 people, if 95% of the marks are identical and only 5% are different, then it is likely that this is a marking error, and its author may be asked for confirmation. By contrast, if an OOI has been annotated differently by half of the panel, the statistical analysis allows the expert responsible for the examination either to draw attention to this OOI or to ask the panel of experts to again analyze this OOI and to debate it. The commanded display mode according to the invention is particularly beneficial in this situation.

Generation of the Preliminary Report

In addition to the benefit of ensuring that no phase of the examination will be forgotten by an expert, using a sequential annotation guide and a terminology database allows the system according to the invention to automatically generate, after the result image, a preliminary examination report that will of course have to be completed by the expert responsible for the examination.

Inspecting the quality of a structure in industry or a medical examination require an examination with various checkpoints. Ideally, in order to ensure the rigor of the inspection, the examination is organized into inspection categories in accordance with a well-defined sequence. Unfortunately, this is not always the case, and the inspector or the doctor fills in his record as he performs his examination. Independently of the skill of the inspector, this leads to difficulties in the traceability and in the reproducibility of the inspection: specifically, some points may be omitted. There may furthermore be a large variation in descriptors of the image depending on the vocabulary of each one, thereby possibly leading to risks of a lack of understanding. Lastly, comparing two examinations performed by different people at different times may be highly tedious or even impossible when the record layouts are not identical.

The system according to the invention also makes it possible to rectify this.

Once the console has generated the result image, it also generates a preliminary examination report comprising an examination layout for the image to be processed in which the responses of each expert are reported and combined sequentially. The layout of the preliminary report and its content are determined by the sequential annotation guide (examination layout and questionnaire) and the terminology database accompanying it, and that were selected (or created) by the administrator when preparing the working session.

Once the result image has been generated, the central unit of the console is programmed to generate, according to the examination layout for the image to be processed, a preliminary examination report comprising the unique identifier of the image to be processed, the result image and the experts' responses to the questionnaire.

The preliminary report preferably also comprises a standardized record of the statistical analysis of the result image. In order words, the preliminary report comprises the results of the statistical analysis in the form of pre-established phrases. For example: “In category A (predefined by the administrator) of the objects of interest, X1 percent of experts found fewer than N1 instances, whereas 100−X1 percent found more of them”, X1 being a real number of between 0 and 100, and N1 being an integer greater than or equal to 0 determined on the basis of the examination. In one exemplary application in the context of bronchoalveolar lavage (“BAL”) analysis with evaluation of the cytological profile that requires numbering of the various cells of the BAL, including those of the macrophages, the preliminary report may comprise a phrase of the type “the analysis shows that the sample comprises X2 percent of macrophages with respect to the total cell population of the analyzed sample”.

In order for the examination to be traceable, the system may make it possible to ascertain who found the most ROIs/OOIs and who found fewer of them.

If the experts respond differently to a question, the preliminary report may comprise, in the chapter on which the question depends, a standardized record in the form of pre-established phrases such as for example: “With regard to the question of ascertaining whether Y (predefined by the administrator) was present, n experts out of N2 responded affirmatively, whereas N2−n responded negatively”, N2 being the total number of experts who participated in the image processing, and n being an integer between 0 and N2.

This is the case for example in the context of a breast cancer. Analyzing a pre-operational biopsy comprises, from the list of questions that will guide the treatment, the question “Number of mitoses in a surface area of **** (predefined by the administrator) mm²”, which will make it possible to establish the mitotic index. The preliminary report may then comprise, in the chapter on which the question depends, a phrase of the type: “Regarding the number of mitoses, the count by n experts out of N2 resulted in a mitotic index of . . . whereas the count by N2−n resulted in a mitotic index of . . . ”.

The system according to the invention is therefore capable of generating a pre-filled-in document, gathering the result of the statistical analyses of the responses and the annotations of the experts, in a predetermined order, without the risk of forgetting the examination or of losing information. However, the preliminary report does not comprise any diagnosis, since it makes do with disclosing the statistical results of the automatic analysis of the annotations of the various experts in a standardized and organized manner.

This preliminary report is then completed by the author responsible for drafting the report.

The standardization of the report and the traceability of the data made possible by the processing system according to the invention make it possible to perform multi-person examinations in order to ensure the relevance and the effectiveness thereof, without the risk of losing information (a risk inherent to a multi-person examination) and with statistical analysis that would be impossible without the system according to the invention.

Specifically, without the system according to the invention, it would be necessary to analyze a considerable amount of data regarding examinations that did not take place at the same time or under the same conditions, based on non-homogeneous records. In addition to the inhibitive amount of work that this represents, the result of such analysis would be highly dubious since the data depend on an excessively large number of parameters.

The system according to the invention makes it possible to reduce the number of these parameters so that the analysis of the examinations is fast, easy and highly meaningful.

Advantageously, the preliminary examination report comprises a unique identifier of the image to be processed, the result image, and the standardized record of statistic analysis of the result image.

According to other embodiments:
The system according to the invention is able to ensure anonymization of the data so that none of the experts know the personal details of the patient whose sample they are examining. For this purpose, the remote server comprises a first memory for storing original images each associated with confidential identification information of the patients, a second memory for storing corresponding images to be processed, without confidential identification information but each associated with a unique identifier, and a table of correspondence between the original images and the images to be processed.
   The deletion of the confidential information, the creation of a unique identifier and the creation of the corresponding data table may be performed automatically by the remote server programmed for this purpose.
   The central unit of the console is programmed to add, to the result image obtained after annotation of an image to be processed, the unique identifier of the image to be processed, and to transmit the result image provided with the unique identifier to the remote server.
   Said remote server may then reassign the personal details to the patient to the result image and to the examination report, so that the doctor responsible for the patient may report the results of the examination to the patient, without the risk of mixing up files, and without the risk of the experts having known the personal details of the patient.
The system according to the invention may also make provision for the console of a first system according to the invention to be able to communicate specifically and securely with a console of another system according to the invention that is installed remotely from the first one and in network communication therewith. This arrangement allows working sessions between two or more groups of remote users (experts, students, etc.).
   The system according to the invention thus comprises at least two consoles for distributing images to be processed and for collecting annotated images, able to communicate with one another, the consoles being programmed to have a settable status allowing the consoles to be organized hierarchically.
   In this case, data is exchanged between at least two consoles while complying with the hierarchical organization of the consoles. Thus, one of the consoles has a primary status, whereas the other consoles have a secondary status, the working session being prepared on the primary console and transmitted to the secondary consoles. During the examination, each console plays its role of centralizing the data of the group on which it depends: it manages the commanded display within the group, generates a result image and statistical analysis of the data of the group.
   The primary console may take control of the secondary consoles, that is to say command them, and allow for example a display commanded by a member of a group for all of the members of all of the groups.
   It may also either generate a result image from the annotated images transmitted directly by the consoles of the other groups, or generate a primary result image from the secondary result images generated and then sent by each of the secondary consoles. This second option has the advantage that the computational power is distributed between all of the consoles in order to generate the final result image. However, this embodiment depends on the quality of the communication between the consoles of the various systems according to the invention. It is still highly beneficial, as the exchanged volume of data is limited. A LoRa or SigFox wireless network could serve as communication network.
The interfaces are advantageously identical, thereby making it possible to ensure viewing homogeneity on the interfaces and reducing diagnosis errors attributable to the hardware. This therefore improves the quality of the examination.
The interfaces which can be used in the system according to the invention may be a mobile terminal, a computer and its screen, a tablet, preferably a touchscreen tablet, a micro-display device situated in the focal plane of the user, a virtual reality headset, etc.

By virtue of the invention, the users overcome the constraints in terms of hardware performance and the speed of their telecommunication network, since the console does not need a third-party network connection in order to operate with the interfaces in local mode.

The system furthermore deals with multiple image formats. By contrast, current items of software operate with a single proprietary image format (that of the scanner manufacturer that markets the software).

Furthermore, the system according to the invention is interoperable with pre-existing laboratory management systems, such as PACSs (for “picture archiving and communication systems” used in hospitals).

The invention has been described primarily with implementation in the medical field. It is absolutely not exclusive to this field, which is determined only by the subject of the images.

The invention may very well be used in the industrial field as a diagnosis assistance or quality inspection tool.

The invention may also apply to the field of automobile rental, as a tool for diagnosis assistance and for observation tracking for vehicle rental.

By virtue of the system, the rental company and the renter each annotate the same image of the vehicle (photo or layout) on their interface (preferably a touchscreen tablet) and the console compares the results with a determined similarity level. If the differences are too great, the observation is not valid, and the participants are invited to compare their annotations and to modify them so that the similarity level is sufficient. In this case, after having generated the result image, the console returns a warning and an image (preferably their processed image) comprising the points of difference on each interface. Advantageously, the console also dynamically displays a similarity value on each interface with a color code: value of the level and formatted in red if the similarity level is insufficient. The participants then agree and modify their processed image. Throughout the annotation modifications, the console interactively and dynamically calculates the similarity level of the two reprocessed images and displays it on each console until the similarity level is sufficient. The console then displays a signal (for example a similarity level in green) and the observation is confirmed.

It is then stored and may be compared with the above observation in the event of a dispute. By virtue of using a sequential annotation guide accompanied by a terminology database and preset annotation marks, the observation is created quickly and reliably, and allows for a reliable log for quickly identifying the responsibilities of each party.

1. A collaborative and interactive image processing system (100), for a field requiring the sharing of opinions and the graphical analysis of images in order to limit the risks of individual errors of judgement, characterized in that it comprises:
a console (100) for distributing images (200) to be processed and for collecting annotated images (201a, 201b, 201c, 201d, 201e), able to communicate locally with at least two processing interfaces (120) that are able to display an image to be processed,
in that the console and the interfaces (120) are programmed:
to transmit a copy of the image to be processed to each processing interface (120) from the distribution console (110), the image to be processed being associated with a type of examination, the type of examination being associated with a list of objects of interest and/or regions of interest, and each objects of interest and/or regions of interest being associated with an annotation mark,
to provide tools (B3, B3a, B3b) for graphically annotating the copy of the image on each interface in order to obtain an image annotated with graphical modifications, the graphical annotation tools being predefined for the image and allowing to graphically define areas of interest and/or add a graphical mark to predefined objects of interest,
to transmit each annotated image to the distribution console (110) from each processing interface (120),
and to generate a result image from the annotated images using a combinatorial algorithm analyzing the graphical modifications of each annotated image in order to determine similar graphical modifications and different graphical modifications between the annotated images, and synthesizing this analysis by generating a result image consisting of pixels of graphical formats that differ between the similar graphical modifications and the different graphical modifications,
and in that the console (110) and the interfaces (120) are programmed:
to allow a display in an individual mode in which the display of each interface is independent of the other interfaces,
and to allow a display in a commanded mode in which the display of a first interface commands the display on the other interfaces, the change between the individual mode and the commanded mode being reversible.

2. The image processing system as claimed in claim 1, wherein the console (110) and the interfaces (120) are programmed to make it possible to mark, on the copy of the image to be processed, at least one region of interest (300, 301, 302, 303) defined by a unique identifier, a shape representative of the region of interest and coordinates in the copy of the image to be processed.

3. The image processing system as claimed in claim 1, comprising:
A data distribution and collection console (110) comprising:
a wireless transceiver (111) for transmitting/receiving a local wireless signal;
a memory for storing at least one image to be processed and at least one result image;
a programmed central unit;
at least two processing interfaces (120) comprising:
a wireless transceiver for transmitting/receiving a local wireless signal compatible with the transceiver of the console for allowing a local exchange of data via the wireless signal;
a memory for storing at least one image to be processed and at least one annotated image;
a programmed central unit.

4. (canceled)

5. The collaborative and interactive image processing system as claimed in claim 3, wherein the central unit of the console (110) and the central unit of each interface (120) are programmed to:
transmit a copy of at least one image to be processed to all of the interfaces;
display a whole image to be processed on each interface,
zoom in and/or zoom out on the displayed image on command and store a zoom value representative of the zoom level of the displayed image with respect to the whole image and a position of the displayed image with respect to the whole image;
execute, from a first interface, a display in a reversible commanded mode of displaying the other interfaces, by commanding a display identical to the display of the first interface on all of the other interfaces;
affix at least one graphical annotation mark on the image to be processed in order to generate an annotated image;
record and transmit each annotated image to the console with an identifier representative of the interface that transmitted the annotated image.

6. The image processing system as claimed in claim 5, wherein, if the display in commanded mode is activated by a user, the central unit of the console and the central unit of each interface are programmed to store an initial display of each interface beforehand, to command a display identical to the display of the first interface on all of the other interfaces, and then to redisplay the initial display of each interface when the commanded mode is deactivated.

7. The image processing system as claimed in claim 3, wherein the central unit of the console is programmed to combine the annotated images sent by each interface and generate a result image with a combinatorial algorithm analyzing the graphical modifications to each annotated image in order to determine similar graphical modifications and different graphical modifications between the annotated images, and synthesizing this analysis by generating a result image consisting of pixels of graphical formats that differ between the similar graphical modifications and the different graphical modifications.

8. The image processing system as claimed in claim 7, wherein the central unit of the console is programmed to generate a result image (400) in which graphical annotation marks (301-302) on the annotated images having a similarity level greater than a determined similarity threshold are displayed in a first graphical format, and in which graphical annotation marks (303, M1, M2, M3, M4) on the annotated images having a similarity level less than the determined similarity threshold are displayed in at least one second graphical format.

9. The image processing system as claimed in claim 8, wherein the central unit of the console is programmed to execute statistical analysis of the graphical annotation marks.

10. The image processing system as claimed in claim 9, wherein the statistical analysis comprises calculating a percentage of graphical annotation marks having a similarity level greater than a determined similarity threshold and calculating a percentage of graphical annotation marks having a similarity level less than the determined similarity threshold.

11. The image processing system as claimed in claim 8, wherein the similarity threshold is settable.

12. The image processing system as claimed in claim 8, wherein the central unit of the console and/or of each interface is also programmed to generate a table of correspondence between each graphical annotation mark, defined at least by position coordinates in the annotated image, and its author.

13. The image processing system as claimed in claim 12, wherein the central unit of the console is also programmed to display an author identifier for each graphical annotation mark on the result image.

14. The image processing system as claimed in claim 3 wherein the central unit of each interface is programmed to be able to be activated with an administrator code or with a user code, the central unit of the console being programmed to display, on an interface activated with the administrator code, administration tools comprising: a list of images to be processed that are present in the memory of the central unit, a list of identifiers of the participants, a list for descriptors of objects or of regions of interest which can be selected by users, each descriptor being associated with a different predefined graphical annotation mark.

15. The image processing system as claimed in claim 14, wherein the console comprises a connection port (112) for connecting to a removable memory connected to the central unit of the console.

16. The image processing system as claimed in claim 14, wherein the console comprises a network connection port connected to the central unit of the console (110), said console being programmed to communicate with a remote server (140) via a communication network in order to download at least one image to be processed and to store it in memory.

17. The image processing system as claimed in claim 16, wherein the remote server (140) comprises a first memory for storing original images each associated with confidential identification information, a second memory for storing corresponding images to be processed, without confidential identification information but each associated with a unique identifier, and a table of correspondence between the original images and the images to be processed.

18. The image processing system as claimed in claim 17, wherein the central unit of the console is programmed to add, to the result image obtained after annotating an image to be processed, the unique identifier of the image to be processed, and to transmit the result image provided with the unique identifier to the remote server.

19. The image processing system as claimed in claim 3, wherein the central unit of the console and the central unit of the interfaces are programmed to display a sequential annotation guide accompanied by a terminology database, to sequentially record the responses of each expert, and to generate a preliminary examination report comprising an examination layout for the image to be processed in which the responses of each expert are reported and combined sequentially.

20. The image processing system as claimed in claim 19, wherein the preliminary examination report comprises a unique identifier of the image to be processed, the result image, and a standardized record of statistical analysis of the result image.

21. The image processing system as claimed in claim 1, comprising at least two consoles (110) for distributing images (200) to be processed and for collecting annotated images (201a, 201b, 201c, 201d, 201e) that are able to communicate with one another, the consoles being programmed to have a settable status allowing the consoles to be organized hierarchically.

22. The image processing system as claimed in claim 21, wherein one of the consoles is programmed to have a primary status, whereas the other consoles have a secondary status, the console having a primary status being able to command the consoles having a secondary status.

23. A collaborative and participative image processing method, for a field requiring the sharing of opinions and the graphical analysis of images in order to limit the risks of individual errors of judgement, characterized in that it comprises the following steps:
placing an image (200) to be processed in a memory of a console (110) able to communicate locally with at least two processing interfaces (120);
transmitting a copy of the image to be processed to each processing interface from the distribution console; the image to be processed being associated with a type of examination, the type of examination being associated with a list of objects of interest and/or regions of interest, and each objects of interest and/or regions of interest being associated with an annotation mark;
on each processing interface, displaying tools (B3, B3a, B3b) for graphically modifying the copy of the image to be processed in order to allow a user to graphically modify the copy of the image to be processed and thus generate an annotated image (201a, 201b, 201c, 201d, 201), the graphical annotation tools being predefined for the image and allowing to graphically define areas of interest and/or add a graphical mark to predefined objects of interest;
allowing a display in an individual mode in which the display of each interface I independent of the other interfaces,
allowing a display in a commanded mode in which the display of a first interface commands the display on the other interface, the change between the individual mode and the commanded mode being reversible,
transmitting each annotated image to the distribution console from each processing interface, and
using the distribution console to generate a result image from the annotated images using a determined combinatorial algorithm analyzing the graphical modifications of each annotated image in order to determine similar graphical modifications and different graphical modifications between the annotated images, and synthesizing this analysis by generating a result image consisting of pixels of graphical formats that differ between the similar graphical modifications and the different graphical modifications.

24. The image processing method as claimed in claim 23, wherein the graphical annotations of the annotated images having a similarity level greater than a determined similarity threshold are marked in a first graphical format in the result image, and wherein the graphical annotations of various images having a similarity level less than the determined similarity threshold are marked in at least one second graphical format in the result image.

25. (canceled)
